(19) United States
(12) Patent Application Publication
(10) Pub. No.: US 2006/0063826 A1
(43) Pub. Date: Mar. 23, 2006

(54) Novel crystal forms of atorvastatin hemi-calcium and processes for their preparation

(76) Inventors: Revital Lifshitz-Liron, Herzlia (IL); Judith Aronhime, Rehovot (IL); Limor Tessler, Natanya (IL)

Correspondence Address: KENYON & KENYON LLP, ONE BROADWAY, NEW YORK, NY, 10004, US

(21) Appl. No.: 11/188,180

(22) Filed: Jul. 22, 2005

Related U.S. Application Data
(60) Provisional application No. 60/590,945, filed on Jul. 22, 2004.

(51) Int. Cl. A61K 31/401

(52) U.S. Cl. 514/423; 548/537

(57) ABSTRACT

Provided are novel crystal forms of atorvastatin hemi-calcium referred to herein as Form XVIII and Form XIX and processes for their preparation and use. Also provided are atorvastatin hemi-calcium acetone solvates.

### CROSS-REFERENCE TO RELATED APPLICATION

This application claims the benefit of provisional applications Ser. No. 60/590,945, filed Jul. 22, 2004, which is incorporated herein by reference.

### FIELD OF THE INVENTION

The present invention relates to crystalline polymorphic forms of atorvastatin hemi-calcium and novel processes for preparing crystalline forms of atorvastatin hemi-calcium.

### BACKGROUND OF THE INVENTION

Atorvastatin,([R—(R*,R*)]-2-(4-fluorophenyl)-P,6-dihydroxy-5-(1-methylethyl)-3-phenyl-4-[(phenylamino)carbonyl]-1H-pyrrole-1-heptanoic acid), depicted in lactone form in formula (I) and its calcium salt of formula (II) are well known in the art, and described inter alia, in U.S. Pat. Nos. 4,681,893, and 5,273,995, which are herein incorporated by reference.

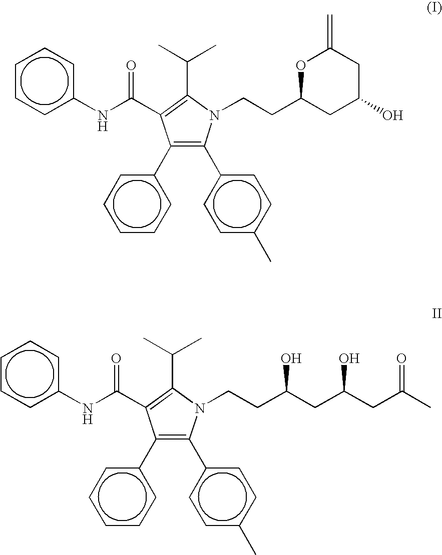

Processes for preparing atorvastatin and its hemi-calcium salt are also disclosed in U.S. Patent Application Publication No. 2002/0099224; U.S. Pat. Nos. 5,273,995; 5,298,627; 5,003,080; 5,097,045; 5,124,482; 5,149,837; 5,216,174; 5,245,047; 5,280,126; Baumann, K. L. et al. Tet. Lett. 1992, 33, 2283-2284, which are hereby incorporated by reference in their entirety and in particular for their teachings related to the preparation of atorvastatin and atorvastatin hemi-calcium.

Atorvastatin is a member of the class of drugs called statins. Statin drugs are currently the most therapeutically effective drugs available for reducing low density lipoprotein (LDL) particle concentration in the blood stream of patients at risk for cardiovascular disease. A high level of LDL in the bloodstream has been linked to the formation of coronary lesions which obstruct the flow of blood and can rupture and promote thrombosis. Goodman and Gilman, The Pharmacological Basis of Therapeutics 879 (9th ed., 1996). Reducing plasma LDL levels has been shown to reduce the risk of clinical events in patients with cardiovascular disease and patients who are free of cardiovascular disease but who have hypercholesterolemia. Scandinavian Simvastatin Survival Study Group, 1994; Lipid Research Clinics Program, 1984a, 1984b.

The mechanism of action of statin drugs has been elucidated in some detail. They interfere with the synthesis of cholesterol and other sterols in the liver by competitively inhibiting the 3-hydroxy-3-methyl-glutaryl-coelizyme A reductase enzyme (“HMG-CoA reductase”). HMG-CoA reductase catalyzes the conversion of HMG to mevalonate, which is the rate determining step in the biosynthesis of cholesterol, and so its inhibition leads to a reduction in the concentration of cholesterol in the liver. Very low density lipoprotein (VLDL) is the biological vehicle for transporting cholesterol and triglycerides from the liver to peripheral cells. VLDL is catabolized in the peripheral cells which releases fatty acids which may be stored in adopcytes or oxidized by muscle. The VLDL is converted to intermediate density lipoprotein (IDL), which is either removed by an LDL receptor, or is converted to LDL. Decreased production of cholesterol leads to an increase in the number of LDL receptors and corresponding reduction in the production of LDL particles by metabolism of IDL.

Atorvastatin hemi-calcium salt trihydrate is marketed under the name LIPITOR® by Pfizer, Inc. Atorvastatin was first disclosed to the public and claimed in U.S. Pat. No. 4,681,893. The hemi-calcium salt depicted in formula (II) is disclosed in U.S. Pat. No. 5,273,995. The '995 patent teaches that the hemi-calcium salt is obtained by crystallization from a brine solution resulting from the transposition of the sodium salt with CaCl₂ and further purified by recrystallization from a 5:3 mixture of ethyl acetate and hexane.

The occurrence of different crystal forms (polymorphism) is a property of some molecules and molecular complexes. A single molecule, like the atorvastatin in formula (I) or the salt complex of formula (II), may give rise to a variety of solids having distinct physical properties like melting point, X-ray diffraction pattern, infrared absorption fingerprint and NMR spectrum. The differences in the physical properties of polymorphs result from the orientation and intermolecular interactions of adjacent molecules (complexes) in the bulk solid. Accordingly, polymorphs are distinct solids sharing the same molecular formula yet having distinct advantageous and/or disadvantageous physical properties compared to other forms in the polymorph family. One of the most important physical properties of pharmaceutical polymorphs is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. On the other hand, where the effectiveness of a drug correlates with peak bloodstream levels of the drug, a property shared by statin drugs, and provided the drug is rapidly absorbed by the GI system, then a more rapidly dissolving form is likely to exhibit increased effectiveness over a comparable amount of a more slowly dissolving form.

Crystalline Forms I, II, III and IV of atorvastatin hemi-calcium are the subjects of U.S. Pat. Nos. 5,959,156 and 6,121,461, assigned to Warner-Lambert. Crystalline atorvastatin hemi-calcium Form V is disclosed in commonly-owned International Publication No. WO 01/36384 (PCT Application No. PCT/US00/31555).

The discovery of new crystalline polymorphic forms of a drug enlarges the repertoire of materials that a formulation scientist has with which to design a pharmaceutical dosage form of a drug with a targeted release profile or other desired characteristic.

### SUMMARY OF THE INVENTION

The present invention provides solid crystalline atorvastatin hemi-calcium acetone solvates.

The present invention further provides a solid crystalline form of atorvastatin hemi-calcium characterized by a powder XRD pattern with peaks at 3.8, 8.0, 8.9 and 10.4±0.2 degrees 2 theta. This form may be an acetone solvate.

The present invention also provides a solid crystalline form of atorvastatin hemi-calcium characterized by a powder XRD pattern with peaks at 3.3, 4.2, 5.6 and 8.2±0.2 degrees 2 theta. This form may be an acetone solvate.

The present invention also provides methods for making the solid crystalline forms described above.

### BRIEF DESCRIPTION OF THE FIGURES

FIG. 1 is a characteristic powder X-ray diffraction pattern of atorvastatin hemi-calcium Form XVIII.

FIG. 2 is a characteristic powder X-ray diffraction pattern of acetone solvate of atorvastatin hemi-calcium Form XIX.

### DETAILED DESCRIPTION OF THE INVENTION

Powder X-ray diffraction (“PXRD”) analysis using a SCINTAG powder X-ray diffiactometer model X'TRA equipped with a solid-state detector. Copper radiation of λ=1.5418 was used. The sample was introduced using a round standard aluminum sample holder with round zero background quartz plate in the bottom.

The present invention provides atorvastatin hemi-calcium salt acetone solvates.

The invention further provides a solid crystalline atorvastatin hemi-calcium, characterized by a powder XRD pattern with peaks at 3.8, 8.0, 8.9 and 10.4±0.2 degrees 2 theta. This solid crystalline atorvastatin hemi-calcium is denominated as Form XVIII.

Atorvastatin form XVIII may be an acetone solvate. Atorvastatin form XVIII may contain up to 1.5% acetone. Preferably, atorvastatin form XVIII may contain up to 1.4% acetone.

Atorvastatin form XVIII can be further characterized by a powder XRD pattern with peaks at 3.0, 18.0, 18.8, 19.6 and 20.6±0.2 degrees 2 theta.

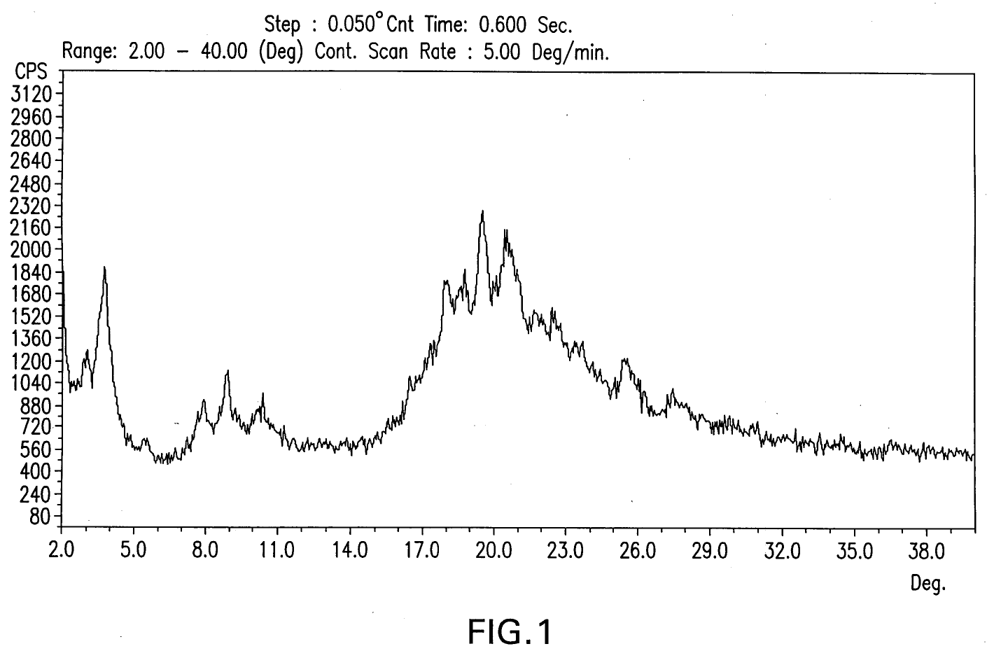

Atorvastatin form XVIII may be further characterized by an XRD pattern substantially as depicted in FIG. 1.

Form XVIII of atorvastatin may be substantially free of crystalline Form I of atorvastatin. In certain embodiments, crystalline atorvastatin hemi-calcium Form XVIII contains less than about 10%, preferably less than about 5%, and even more preferably less than about 1% (by weight) of atorvastatin hemi-calcium Form I.

Another aspect of the present invention is a process for preparing Atorvastatin Form XVIII. The method of preparing crystalline atorvastatin hemi-calcium Form XVIII comprises:
(a) dissolving atorvastatin hemi-calcium in acetone to form a solution;
(b) maintaining the solution until a precipitate is obtained; and
(b) recovering the precipitate.

Preferably, the atorvastatin hemi-calcium of step (a) is Form V. Preferably, step (b) comprises stirring at about room temperature for about 40 hours to about 70 hours. Preferably, the recovering in step (c) comprises filtering and drying the precipitate.

The present invention further provides a solid crystalline atorvastatin hemi-calcium, characterized by a powder XRD pattern with peaks at 3.3, 4.2, 5.6 and 8.2±0.2 degrees 2 theta. This solid crystalline atorvastatin hemi-calcium is denominated as Form XIX.

Atorvastatin form XIX can be further characterized by a powder XRD pattern with peaks at 17.0, 19.2 and 22.0±0.2 degrees 2 theta.

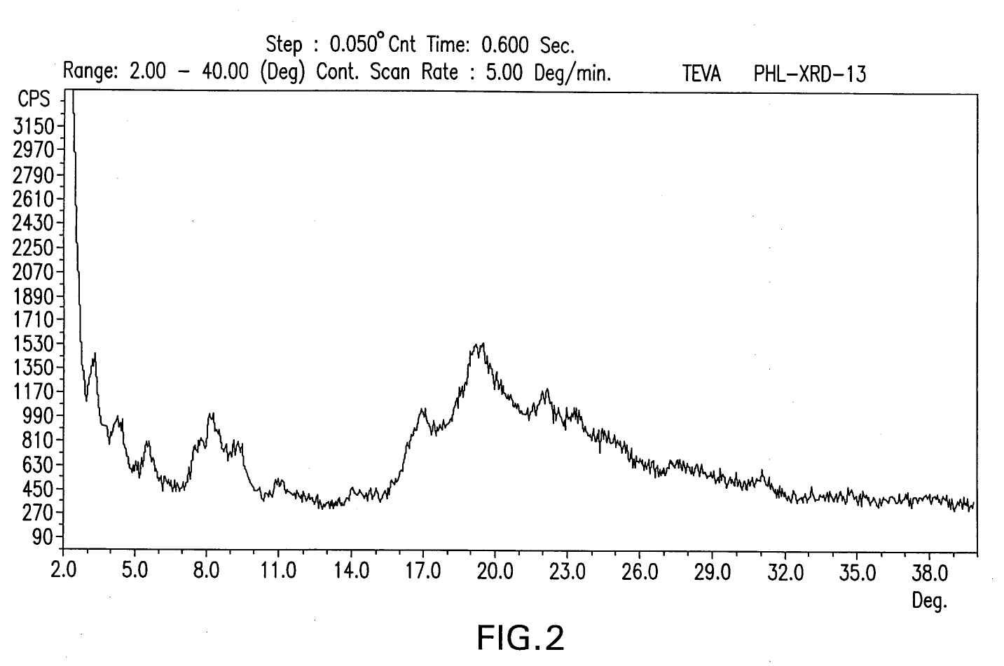

Atorvastatin Form XIX may be further characterized by an XRD pattern as substantially depicted in FIG. 2.

Atorvastatin Form XIX may be an acetone solvate. Atorvastatin Form XIX may contain up to 6.0% acetone. Preferably, atorvastatin Form XVIII may contain up to 5.9% acetone.

Form XIX of atorvastatin may be substantially free of crystalline Form I of atorvastatin. In certain embodiments, crystalline atorvastatin hemi-calcium Form XIX contains less than about 10%, preferably less than about 5%, and even more preferably less than about 1% (by weight) of atorvastatin hemi-calcium Form I.

Another aspect of the present invention is a process for preparing Atorvastatin Form XIX. A method of preparing crystalline atorvastatin hemi-calcium Form XIX comprises performing a scaled-up process for preparing Form XVIII. Preferably, the amount of atorvastatin hemi-calcium and acetone are scaled-up by a factor of about 4 to about 8. More preferably, the amount of atorvastatin hemi-calcium and acetone are scaled-up by a factor of about 6. It is within the skill of those of ordinary skill in the art, guided by the present disclosure, to choose the appropriate amount of atorvastatin hemi-calcium and acetone to obtain the desired crystalline form, either Form XVIII or Form XIX, with the use of, at most, only routine experimentation.

Atorvastatin hemi-calcium solid crystalline Forms XVIII and XIX are useful for reducing the plasma low density lipoprotein level of a patient suffering from or susceptible to hypercholesterolemia. For this purpose, Form XVIII or Form XIX will typically be administered to human patients in a unit dose of from about 0.5 mg to about 100 mg. For most human patients, a dose of from about 2.5 to about 80 mg per day, more particularly from about 2.5 to about 20 mg per day, causes a lowering of the plasma low density lipoprotein level. Whether such lowering is sufficient or whether the dose or dose frequency should be increased is a determination that is within the skill level of appropriately trained medical personnel.

In another aspect, the invention provides compositions and dosage forms comprising the forms of atorvastatin hemi-calcium solvate and their mixtures. The compositions of the invention include powders, granulates, aggregates and other solid compositions comprising Forms XVIII and/or XIX of atorvastatin hemi-calcium solid crystalline. In addition, Forms XVIII and XIX solid compositions that are contemplated by the present invention may further include diluents, such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents like calcium carbonate and calcium diphosphate and other diluents known to the pharmaceutical industry. Yet other suitable diluents include waxes, sugars and sugar alcohols like mannitol and sorbitol, acrylate polymers and copolymers, as well as pectin, dextrin and gelatin.

Further excipients that are within the contemplation of the present invention include binders, such as acacia gum, pregelatinized starch, sodium alginate, glucose and other binders used in wet and dry granulation and direct compression tableting processes. Excipients that also may be present in a solid composition of Forms XVIII and XIX atorvastatin hemi-calcium further include disintegrants like sodium starch glycolate, crospovidone, low-substituted hydroxypropyl cellulose and others. In addition, excipients may include tableting lubricants like magnesium and calcium stearate and sodium stearyl fumarate; flavorings; sweeteners; preservatives; pharmacy parenteral (including subcutaneous, intramuscular, and intravenous), inhalant and ophthalmic administration. Although the most suitable route in any given case will depend on the nature and severity of the condition being treated, the most preferred route of the present invention is oral. Dosages may be conveniently presented in unit dosage form and prepared by any of the methods well-known in the art of pharmacy.

Dosage forms include solid dosage forms, like tablets, powders, capsules, suppositories, sachets, troches and losenges as well as liquid suspensions and elixirs. While the description is not intended to be limiting, the invention is also not intended to pertain to true solutions of atorvastatin hemi-calcium whereupon the properties that distinguish the solid forms of atorvastatin hemi-calcium are lost. However, the use of the novel forms to prepare such solutions (e.g. so as to deliver, in addition to atorvastatin, a solvate to said solution in a certain ratio with a solvate) is considered to be within the contemplation of the invention.

Capsule dosages, of course, will contain the solid composition within a capsule which may be made of gelatin or other conventional encapsulating material. Tablets and powders may be coated. Tablets and powders may be coated with an enteric coating. The enteric coated powder forms may have coatings comprising phthalic acid cellulose acetate, hydroxypropylmethyl-cellulose phthalate, polyvinyl alcohol phthalate, carboxymethylethylcellulose, a copolymer of styrene and maleic acid, a copolymer of methacrylic acid and methyl methacrylate, and like materials, and if desired, they may be employed with suitable plasticizers and/or extending agents. A coated tablet may have a coating on the surface of the tablet or may be a tablet comprising a powder or granules with an enteric-coating.

Preferred unit dosages of the pharmaceutical compositions of this invention typically contain from 0.5 to 100 mg of one of the novel atorvastatin hemi-calcium Forms XVIII and XIX, or mixtures thereof, or mixtures with other forms of atorvastatin hemi-calcium. More usually, the combined weight of the atorvastatin hemi-calcium forms of a unit dosage are from 2.5 mg to 80 mg.

The crystalline forms of the present invention used to prepare pharmaceutical formulations may be substantially pure with respect to other crystalline forms, i.e., the pharmaceutical formulations may contain less than about 10%, preferably less than about 5%, and even more preferably less than about 1% (by weight) of other crystalline forms of atorvastatin hemi-calcium. In particular, the pharmaceutical formulations comprising Form XVIII may contain less than about 10%, preferably less than about 5%, and even more preferably less than about 1% (by weight) of Form I. The pharmaceutical formulations comprising Form XIX may contain less than about 10%, preferably less than about 5%, and even more preferably less than about 1% (by weight) of Form I. In certain embodiments, the pharmaceutical formulations may contain less than about 10%, preferably less than about 5%, and even more preferably less than about 1% (by weight) of amorphous atorvastatin.

Alternatively, pharmaceutical formulations of the present invention may also contain one or both of Form XVIII or Form XIX in a mixture with other forms of atorvastatin. However, it is preferred that the pharmaceutical formulations or compositions of the present invention contain 25-100% by weight, especially 50-100% by weight, of at least one of Form XVIII or Form XIX, based on the total amount of atorvastatin in the formulation or composition. Preferably, such an amount of the novel Form XVIII or Form XIX of atorvastatin hemi-calcium is 75-100% by weight, especially 90-100% by weight. Highly preferred is an amount of 95-100% by weight.

As used herein, “room temperature” or “RT” is meant to indicate a temperature of about 18-25° C., preferably about 20-22° C.

“Therapeutically effective amount” means the amount of a crystalline form that, when administered to a patient for treating a disease or other undesirable medical condition, is sufficient to have a beneficial effect with respect to that disease or condition. The “therapeutically effective amount” will vary depending on the crystalline form, the disease or condition and its severity, and the age, weight, etc., of the patient to be treated. Determining the therapeutically effective amount of a given crystalline form is within the ordinary skill of the art and requires no more than routine experimentation.

Certain processes of the present invention involve crystallization out of a particular solvent. One skilled in the art would appreciate that the conditions of crystallization often can be modified somewhat without affecting the crystalline form obtained. For example, when mixing atorvastatin hemi-calcium in a solvent to form a solution, warming of the mixture may be desirable to completely dissolve the starting material. If warming does not clarify the mixture, the mixture may be diluted or filtered. To filter, the hot mixture may be passed through paper, glass fiber, or other membrane material, or a clarifying agent such as celite. Depending upon the equipment used and the concentration and temperature of the solution, the filtration apparatus may need to be preheated to avoid premature crystallization.

The conditions may also be changed to induce precipitation. A preferred way of inducing precipitation is to reduce the solubility of the solvent. The solubility of the solvent may be reduced, for example, by cooling the solvent. Precipitation may also be induced by evaporating some of the solvent or by adding an anti-solvent.

The crystalline forms of the present invention may be distinguished by their PXRD patterns. The crystalline forms have characteristic PXRD peak positions in the range of 2-40 degrees two theta. According to these characteristic peak positions, the skilled artisan can identify the crystalline forms and also identify and quantify their crystalline form impurities.

One skilled in the art would appreciate that there is a small amount of uncertainty involved in PXRD measurements, generally of the order of about ±0.2 degrees 2 theta for each peak. Accordingly, PXRD peak data herein are presented in the form of “a PXRD pattern with peaks at A, B, C, etc. ±0.2 degrees 2 theta.” This indicates that, for the crystalline form in question, the peak at A could, in a given instrument on a given run, appear somewhere between A ±0.2 degrees 2 theta, the peak at B could appear at B ±0.2 degrees 2 theta, etc. Such small, unavoidable uncertainty in the identification of individual peaks does not translate into uncertainty with respect to identifying individual crystalline forms since it is generally the particular combination of peaks within the specified ranges, not any one particular peak, that serves to unambiguously identify crystalline forms.

The particle size distribution (PSD) of the active ingredient is one of the key parameters of a formulation. For measuring particle size, the following main methods may be employed: sieves, sedimentation, electrozone sensing (coulter counter), microscopy, Low Angle Laser Light Scattering (LALLS). The new forms of the invention have a preferred maximum particle size of 500 μm. Preferably, the particle size is less than 300 μm, less than 200 μm, less than 100 μm, or even less than 50 μm.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

### Example 1

### Procedure for Preparing Form XVIII

A slurry of Atorvastatin hemi-calcium salt crystal Form V (10 g) in Acetone (70 ml) was stirred at room temperature for 8 hours to obtain complete dissolution. The obtained solution was stirred at room temperature for an additional 40 hours to obtain a massive precipitant. Acetone (280 ml) was added in order to dilute the slurry. The product was isolated by filtration and dried in a vacuum oven at 40° C. for 20 hours to obtain 6.6 g of Atorvastatin hemi-calcium salt crystal Form XVIII. The level of acetone was 13890 ppm (1.4%).

### Example 2

### Procedure for Preparing Form XIX

A slurry of Atorvastatin hemi-calcium salt crystal Form V (60 g) in Acetone (420 ml) was stirred at room temperature for 8 hours to obtain complete dissolution. The obtained solution was stirred at room temperature for an additional 64 hours to obtain a massive precipitant. The product was isolated by filtration, washed with Acetone (4×250 ml) and dried in a vacuum oven at 40° C. for 21 hours to obtain 59.4 g of Atorvastatin hemi-calcium salt crystal Form XIX. The level of acetone was 58695 ppm (5.9%).

What is claimed is:

1. Atorvastatin hemi-calcium salt acetone solvate.

2. Crystalline atorvastatin hemi-calcium characterized by a PXRD pattern having peaks at 3.8, 8.0, 8.9, and 10.4±0.2 degrees 2 theta.

3. The crystalline atorvastatin hemi-calcium of claim 2, further characterized by PXRD peaks at 3.0, 18.0, 18.8, 19.6 and 20.6±0.2 degrees 2 theta.

4. The crystalline atorvastatin hemi-calcium of claim 2, having a PXRD spectrum substantially as depicted in FIG. 1.

5. The crystalline atorvastatin hemi-calcium of claim 2 that is an acetone solvate.

6. The crystalline atorvastatin hemi-calcium of claim 5 containing up to about 1.5% acetone.

7. The crystalline atorvastatin hemi-calcium of claim 6 containing up to about 1.4% acetone.

8. The crystalline atorvastatin hemi-calcium of claim 2 which contains less than about 10% (by weight) of atovastatin hemi-calcium Form I.

9. The crystalline atorvastatin hemi-calcium of claim 8 which contains less than about 5% (by weight) of atovastatin hemi-calcium Form I.

10. The crystalline atorvastatin hemi-calcium of claim 9 which contains less than about 1% (by weight) of atovastatin hemi-calcium Form I.

11. Crystalline atorvastatin hemi-calcium characterized by a PXRD pattern having peaks at 3.3, 4.2, 5.6, and 8.2±0.2 degrees 2 theta.

12. The crystalline atorvastatin hemi-calcium of claim 11, that is further characterized by PXRD peaks at 17.0, 19.2 and 22.0±0.2 degrees 2 theta.

13. The crystalline atorvastatin hemi-calcium of claim 11, having a PXRD spectrum substantially as depicted in FIG. 2.

14. The crystalline atorvastatin hemi-calcium of claim 11 that is an acetone solvate.

15. The crystalline atorvastatin hemi-calcium of claim 14, containing up to about 6.0% acetone.

16. The crystalline atorvastatin hemi-calcium of claim 15, containing up to about 5.9% acetone.

17. The crystalline atorvastatin hemi-calcium of claim 11, which contains less than about 10% (by weight) of atovastatin hemi-calcium Form I.

18. The crystalline atorvastatin hemi-calcium of claim 17 which contains less than about 5% (by weight) of atovastatin hemi-calcium Form I.

19. The crystalline atorvastatin hemi-calcium of claim 18 which contains less than about 1% (by weight) of atovastatin hemi-calcium Form I.

20. A method of preparing crystalline atorvastatin hemi-calcium characterized by a PXRD pattern having peaks at 3.8, 8.0, 8.9, and 10.4±0.2 degrees 2 theta comprising:
(a) dissolving atorvastatin hemi-calcium in acetone to form a solution;
(b) maintaining the solution until a precipitate is obtained; and
(c) recovering the precipitate.

21. The method of claim 20, wherein step (b) comprises stirring for about 40 to about 70 hours.

22. The method of claim 20, wherein the temperature is about room temperature.

23. A method of preparing crystalline atorvastatin hemi-calcium characterized by PXRD peaks at 3.3, 4.2, 5.6 and 8.2±0.2 degrees 2 theta comprising performing the process of claim 20, wherein the amount of the atorvastatin hemi-calcium and acetone are scaled-up by a factor of about 4 to about 8.

24. The method of claim 23, wherein the amount of the atorvastatin hemi-calcium and acetone are scaled-up by a factor of about 6.

25. A method of preparing crystalline atorvastatin hemi-calcium Form XVIII or Form XIX comprising:
(a) dissolving atorvastatin hemi-calcium in acetone to form a solution;
(b) maintaining the solution until a precipitate is obtained; and
(c) recovering the precipitate.

26. The method of claim 25 wherein the ratio of atorvastatin to acetone in step (a) is about 1 g:7 ml.

27. The method of claim 26 wherein the amount of atorvastatin dissolved in step (a) is adjusted so as to produce a precipitate of atorvastatin hemi-calcium Form XVIII in step (b).

28. The method of claim 27 wherein the amount of atorvastatin dissolved in step (a) is about 10 g.

29. The method of claim 25 wherein the amount of atorvastatin dissolved in step (a) is adjusted so as to produce a precipitate of atorvastatin hemi-calcium Form XIX in step (b).

30. The method of claim 29 wherein the amount of atorvastatin dissolved in step (a) is about 60 g.

31. A pharmaceutical composition prepared by combining at least one pharmaceutically acceptable excipient with at least one of the crystalline forms of atorvastatin hemi-calcium, of any of claims 2 and 11.

32. A method of treating a patient with hypercholesterolemia or hyperlipidemia comprising administering to the patient a therapeutically effective amount of the pharmaceutical composition of claim 31.
